(19) United States
(12) Patent Application Publication
(10) Pub. No.: US 2007/0255215 A1
(43) Pub. Date: Nov. 1, 2007

(54) Long-term central venous catheter device

(76) Inventor: Marcos Augusto Silva Pires E Albuquerque, Rio de Janeiro (BR)

Correspondence Address: ARENT FOX PLLC, 1050 CONNECTICUT AVENUE, N.W., SUITE 400, WASHINGTON, DC, 20036, US

(21) Appl. No.: 11/785,714

(22) Filed: Apr. 19, 2007

(30) Foreign Application Priority Data
Apr. 26, 2006 (BR) PI 0602271-5

(51) Int. Cl. A61M 37/00

(52) U.S. Cl. 604/131

(57) ABSTRACT

The present invention refers to a new device to be implanted in a human being, and that once connected to a distal extremity of a catheter tube placed in the central venous system, allows needleless I.V delivery, minimizing workers exposure against needlestick injuries, and a painless manipulation for the patients. Yet, as it is constructed detached from the catheter's tube, allows to the surgeons accomplish the procedure through a retrograde technique which enlarge implantation's success when compared with the one-piece model of the Semi-Implantable Catheters' type which are currently available, that is novelty in the art.

The present invention refers to a new device to be implanted in a human being that once being connected to a catheter tube positioned inside the venous system, allows of drugs administration by a long-time without needles' use. It is applied mainly in the area of oncology, and is conceived to the all patients that require long-term central venous accesses.

### BACKGROUND OF THE INVENTION

It is well known that some medications need to be applied in vein for a long period of time. Mainly in the oncologic area the medications must be applied directly in the central venous system through these Catheters called of Long-Term Devices. They are the link between the external environment and the intravascular environment, being used by several months or even years whenever requested, interpolated with long idle periods between each manipulation.

The Long-Term Catheters available until now have configurations qualified as Semi-Implantable or Totally-Implantable.

The first one (Semi-Implantable), has similar medical indication with the invention, but has substantial differences in the way of catheter-bearing and in the implantation's technique.

The current Long-Term Semi-Implantable Catheter is a one-piece catheter which exits through the skin in a long external length to be connected straight into a syringe, or in the I.V tubing, without needles' use. However, this long length requires constant dressing being unpleasant mainly to patients with sensitive skin. That provokes daily restrictions like shower baths, once the external segment coiled and attached over the chest, is a considerable discomfort. Also, to accomplish a surgical implant, a one-piece catheter restricts the technical options to a surgeon overcome anatomical challenging.

The second type (called totally-implantable), is a long-term catheter that remains totally inlayed at the human body. It is a two-piece configuration, therein similar to the technical disclosure here required, but different in the practice, once it stays completely positioned under the skin in which a needle is necessary to achieve its interior and enable the drugs be delivered.

### SUMMARY OF THE INVENTION

It is ideal for patients who need cancer treatment like bone marrow transplantations and hematological malignancies like leukemia, lymphomas and myelomas, as well as other less common diseases where the skin puncture must be avoided or when is necessary a rate of high venous flow like hemodialysis.

The present invention has many purposes. First it allows intravenous medication delivery without needles reducing the contamination risk, and needlestick injuries, at the medical staff. Second, it provides more comfort to the patient by avoiding undesirable and painful needle punctures, as well as it dispense those uncomfortable dressings daily-made. Third, it has a clean site of manipulation that besides other benefits prevents contamination and allows patients to shower. Fourth, whether compared with a totally-implantable device, beyond needleless manipulation it also permits a much higher flow rate, which is essential for the treatments that are indicated. Last, but not least, broaden the surgical technical alternatives face to anatomical challenging situations, once the presented invention device compounds a two-piece catheter (what helps the surgeon), whereas the current semi-implantable is a one-piece configuration.

The goal of the invention is based on the fact that it is an device “almost” totally-implantable, which assumes a semi-implantable configuration because part is inlayed in the human body and other part stays outside, what is a invention novelty once it offers venous approach without needle manage.

To differ from current Semi-Implantable Catheters, this technical disclosure argue that it is a two-piece configuration, what is essential to surgical implant techniques (anatomical challenging conditions), and which avoid the coiled catheter's tube over the chest, so unpleasant for the patient's bearing a Long-Term Catheter.

To differ from current Totally-Implantable Catheters it argue be an device partially (or almost) implantable, being able to avoid handling by needles and permits a high flow venous delivery rate, once it has an internal diameter larger than a needle.

### DESCRIPTION OF THE DRAWINGS

FIG. 1 illustrates a perspective view of the device of the present invention;

FIG. 2 shows the internal components of the invention;

FIG. 3 illustrates a cut view of the device using another example as blocking, a passive system, like a fissure.

FIG. 4 shows the draw of the final aspect of the device applied to a human body.

### DETAILED DESCRIPTION OF THE INVENTION

The invention device can be built in two models. The single, developed in a single set (used to single-lumen catheter), and the double, developed in two identical set fixed side-by-side (used in dual-lumen catheter).

In the present invention, the double model is particularly described, where one set fasten another as twins, allowing handling each one separately, but at the same time. The single model is the same system being built as single set.

The invention devices are always characterized by a closed system and for that reason they need a sort of valve sealing the system, however this does not imply a specific object, because many already known systems can be used if they suit this purpose.

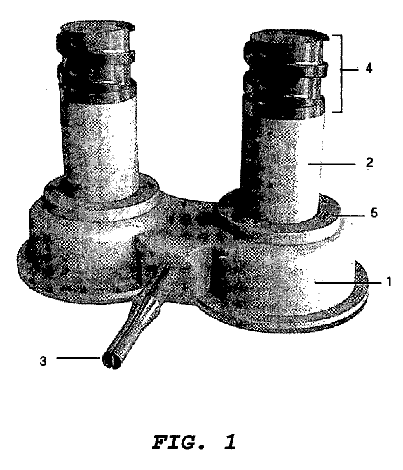

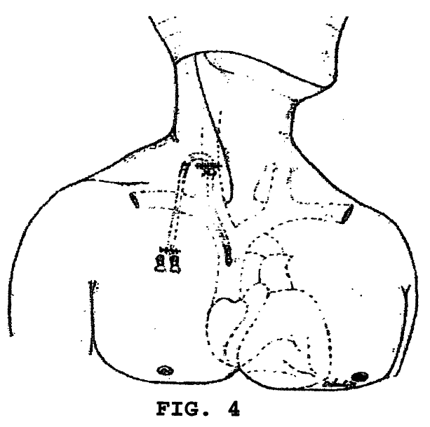

The structure of the invention's device is made of an inferior part composite by a reservoir named Tank (1)—FIG. 1—which when it is surgically implanted it stays positioned under the skin. Another superior tubular part structure, in a tower's shape (2) complement the body of the device and will be positioned on the outside, transposing the skin as shown in FIG. 4.

In the double model, both set are “welded” each other sidelong, forming an assemblage of one-piece. From the bottom of the tank (1) there are two semicircular tubes to medications outlet (3)—FIG. 1—which will be connected to the dual-lumen catheter. In case of the single-model, the dual-lumen tube is substituted by the single-lumen.

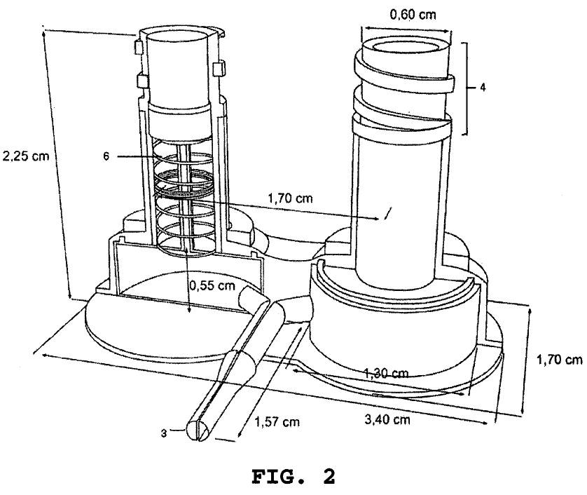

The structure of the device is externally covered by a silicon medical standard rubber, letting nude just the top of the tower, (4) where there is a connector of luer-lock to be attached to the I.V tubing as shown in the FIGS. 1 and 2 and identified with the number (4), and the outlet tube (to be attached to the catheter) indicated with a number (3).

Around the tower bottom (2), lie down over the tank (1), a band is fixed (5) to adhere to the device at the skin's internal layer, aiming to protect against infections and rejections; the preferred material for the band is Dacron® with 1 mm thickness.

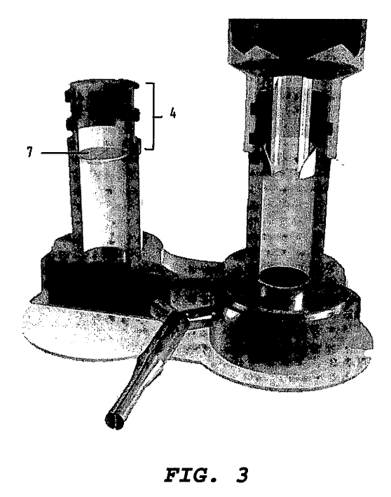

The internal structure of the invention's device has been made in a rigid material, preferably titanium. Inside the tower (2) always will have a locking valve system (that can be in many different models and that is not the point of this report, once it is obvious) for separating the internal and external environments. In the present invention we have detailed two possible ways to be created a valve, one with a system called “active”, made by a spring (6)—FIG. 2—and another system called “passive”, made from the proper silicon rubber (7)—FIG. 3—both built inside the tower (2), in a way that they can block the communication with the external environment during idle time.

For the system to work, a syringe or an I.V tubing, is attached directly over the tower (4) (FIG. 1,2,3). When this occurs the valve system opens (pushed downward) not needing to use needles (FIG. 3). In FIG. 2 is also shown the approximate size of the present invention.

Referring to FIGS. 2 and 3, the device having a longitudinal cut with the internal structures, which are the towers and the two valves' examples for closing the system, is to be considered that the valve's system here showed doesn't interfere on the invention's purpose. The goal of the invention is that the device is “almost” totally-implantable, allowing it to be manipulated without needles, unlike the totally-implanted type, where the whole device is under the skin needing needle to be accessed.

This invention is not limited to the description given, and should be understood in its broader purpose. The models can have several diameters, depending on the treatment the patient needs.

Many changes and representations of the invention will come to the mind of those who have the skills to use it, and they will have the benefit of the teachings shown in the previous descriptions. Plus, it should be understood that the invention is not limited to the specific form it was presented, and that modifications and other forms are included in the same purpose of the attached claims. Although specific terms were used here, they were used only in a generic and descriptive form and not with the purpose of limitation.

1- Long-term central venous catheter device which is semi-implantable (or almost totally-implantable) and holds at least one tank element (1) in combination with at least one tower element (2) where the tank element receives the catheter tube (3); where the structure of the device is recovered with a medical standard silicon revetment that does not involve the superior portion (4) of the tower (2) and the outlet tube (3); internally the device holds a system of valve-locking (6 or 7, as examples) for connections.

2- The device, according to claim 1, which comprises a band (5) of fibrotic adherence.

3- Device, according to claim 1, wherein the connection between the internal and external means of the human body is done by a luer-lock system (4), sealed by a valve system (6 or 7, as an example) positioned inside the tower (2).

4- The device, according to claim 1, which is connected directly to a catheter whose the tip must be positioned in the interior of the Superior Vena Cava.
